(19) United States
(12) Patent Application Publication
(10) Pub. No.: US 2024/0167047 A1
(43) Pub. Date: May 23, 2024

(54) USE OF INDICA RICE OsRNR10 GENE

(71) Applicant: NANJING AGRICULTURAL UNIVERSITY, Nanjing, Jiangsu (CN)

(72) Inventors: Shan LI, Nanjing (CN); Yunzhi HUANG, Nanjing (CN); Chengbo SHEN, Nanjing (CN); Yaojun QIN, Nanjing (CN); Siyu ZHANG, Nanjing (CN)

(73) Assignee: NANJING AGRICULTURAL UNIVERSITY, Nanjing, Jiangsu (CN)

(21) Appl. No.: 18/475,615

(22) Filed: Sep. 27, 2023

(30) Foreign Application Priority Data
Nov. 23, 2022 (CN) 202211470272.9

(51) Int. Cl. C12N 15/82; C12Q 1/6895

(52) U.S. Cl. CPC C12N 15/8227; C12N 15/8205; C12Q 1/6895

(57) ABSTRACT

Use of an indica rice OsRNR10 gene in enhancing rice root system development, and improving nitrogen use efficiency and yield, where sequences of the indica rice OsRNR10 gene are shown in SEQ ID NOs: 1 and 2. 82 IRAT261-carrying single segment substitution lines under the background of Huajingxian 74 are utilized to carry out a quantitative trait locus (QTL) analysis. A nitrogen-response root system development regulatory factor OsRNR10 is further finely mapped. The indica rice haplotype OsRNR10 reduces the expression level, and enables indica rice to have a better root system architecture and a higher nitrate nitrogen uptake rate. By introducing an excellent allelic variation of OsRNR10 in the indica rice into japonica rice, the root system development of the japonica rice can be enhanced, and the nitrogen use efficiency and yield of the japonica rice can be improved.

The present application contains a Sequence Listing that has been submitted electronically and is hereby incorporated by reference herein in its entirety. The electronic Sequence Listing, created on Sep. 21, 2023, is named 226521_Sequence Listing.xml and is 19,766 bytes in size.

### TECHNICAL FIELD

The present invention belongs to the technical field of plant genetic engineering. Specifically, the present invention relates to use of OsRNR10 in regulating and controlling rice root system development, nitrogen use efficiency and yield, and alleles of indica rice.

### BACKGROUND

Rice (Oryza sativa L.) is one of the most important food crops in China. The planting area and the yield of the rice rank second among the food crops. Therefore, green and safe production of rice plays a vital role in the food safety of China.

Data of Food and Agricultural Organization of the United Nations (FAO) shows that the consumption of a nitrogen fertilizer worldwide has increased significantly in the past decade, but the crop yield has increased slowly. The excessive input of the nitrogen fertilizer has not greatly increased the yield, but led to a decline in economic and ecological benefits. How to continuously improve the crop yield under the condition of reducing the fertilizer input becomes a major problem to be urgently solved for guaranteeing the food safety and the sustainable development of agriculture.

The plastic development of a plant root system is mainly influenced by the combination of internal hormone signals and external environmental factors. Plant hormones play an important role in regulating and controlling the early establishment of roots and the postembryonic growth. The joint regulation and control of multiple hormones endows the growth and development of the plant root system with flexibility and plasticity, where auxin plays an important role in the plastic development of the root system (Roychoudhry et al., 2022). In order to maintain the growth and development of plants, the plant root system needs to absorb 14 necessary mineral elements from the soil, where nitrogen is one of the most important nutrients. The level of an external nitrogen source is an important factor limiting the plant root system development. The nitrogen use efficiency (NUE) of crops is inherently complex but largely depends on plant N uptake efficiency (NUpE) of plants (Masclaux-Daubresse et al., 2022). Since the root is the major nitrogen uptake organ, the NUpE is affected by the root system architecture (RSA). Besides, the external nitrogen source greatly affects the RSA of plants. Generally, the low nitrogen condition leads to the stronger RSA to ensure that the plants maximally acquire the external nitrogen source under the low nitrogen condition (Jia et al., 2022).

The current understanding of the coordinated regulation and control of plant growth and development by nitrogen and auxin mainly derives from the study of a model plant Arabidopsis thaliana, and most focuses on the regulation and control of the RSA. On one hand, the external nitrogen source can change the RSA of the Arabidopsis thaliana by regulating and controlling the expressions of an auxin synthesis gene TAR2 (Tryptophan Aminotransferase Related 2) and an auxin receptor gene AtAFB3 (Auxin-signaling F-box Protein 3) (Ma et al., 2014; Vidal et al., 2010). On the other hand, under condition of low-concentration nitrate nitrogen (NO₃⁻), a nitrate nitrogen transporter NRT1.1 transports auxin to a base part, resulting in a decrease in the accumulation amount of auxin in a lateral root tip and thereby inhibiting lateral root growth (Krouk et al., 2010). However, the genetic mechanism of an auxin pathway is significantly different between monocots and dicots (Wang et al., 2018). How the nitrogen and auxin interact to influence the rice root system development and further synergistically regulate and control the NUE and yield are not clear.

### SUMMARY

The objective of the present invention is to provide use of an indica rice OsRNR10 gene aiming at the shortcomings of the prior art.

Another objective of the present invention is to provide a marker related to plasticity development of a rice root system and nitrogen use efficiency of rice.

Yet another objective of the present invention is to provide a method for improving rice root system development and nitrogen use efficiency.

The objectives of the present invention can be realized by the following technical solutions:

Provided is use of an indica rice OsRNR10 gene in optimizing rice root system development, and improving rice nitrogen use efficiency and yield, where a sequence of an indica rice OsRNR10 gene promoter is shown in SEQ ID NO: 1, a gDNA nucleotide sequence is shown in SEQ ID NO: 2, a cDNA nucleotide sequence is shown in SEQ ID NO: 3, and an encoded protein amino acid sequence is shown in SEQ ID NO: 4.

In japonica rice IRAT261, a sequence of OsRNR10 gene promoter is shown in SEQ ID NO: 5, a gDNA nucleotide sequence is shown in SEQ ID NO: 6, a cDNA nucleotide sequence is shown in SEQ ID NO: 7, and an encoded protein amino acid sequence is shown in SEQ ID NO: 8.

Provided is use of an indica rice OsRNR10 gene promoter in optimizing rice root system development, and improving rice nitrogen use efficiency and yield, where a sequence of the indica rice OsRNR10 gene promoter is shown in SEQ ID NO: 1.

Provided is a marker related to nitrogen-dependent regulation and control of rice root system development and rice nitrogen use efficiency, where the marker is a 3,496-bp sequence of 912nd-4,408th bp in an OsRNR10 gene promoter shown in SEQ ID NO: 5; the marker exists in japonica rice, causes a high expression of OsRNR10, weakens root system development, and enables a root system to be insensitive to an external nitrogen source change and have a slow uptake rate of NO₃⁻; and the marker is deleted in indica rice, causes a low expression of the OsRNR10, and enables the root system to be more developed and more sensitive to the external nitrogen source change, and have an increased NO₃⁻ uptake rate.

Provided is a method for determining rice root system development and nitrogen use efficiency, where when a rice OsRNR10 gene promoter is detected, if the marker of the present invention exists, the rice variety root system development is relatively weak, and the nitrogen use efficiency is low; and if the marker of the present invention is deleted, the rice variety root system development is relatively strong, and the nitrogen use efficiency is high.

Preferably, sequences of primers in detecting the rice OsRNR10 gene promoter are shown in SEQ ID NO: 9 and SEQ ID NO: 10.

Provided is a method for enhancing rice root system development and nitrogen use efficiency, where an OsRNR10 gene in rice is knocked out or silenced, and the rice is preferably japonica rice. The method of the present invention can improve nitrogen use efficiency and yield of rice by optimizing the root system architecture of the rice.

OsRNR10 promotes its stability through a mono-ubiquitination modification of a K53 site of an auxin synthesis inhibiting factor OsDNR1 (DULL NITROGEN RESPONSE 1), and further inhibits auxin accumulation, root system growth, and uptake of NO₃⁻.

### Beneficial Effects

The present invention utilizes 82 single segment substitution line materials (chromosome segment substitution lines, CSSLs) under the background of Huajingxian 74 (HJX74) to carry out root system size comparison analysis under the treatment of different nitrogen concentrations, maps a nitrogen-response root system development regulatory factor OsRNR10 on chromosome 10 (REGULATOR OF N-RESPONSIVE RSA ON CHROMOSOME 10) in combination with a map-based cloning method, and further discovers through studies that OsRNR10 promotes its stability through a mono-ubiquitination modification of a K53 site of an auxin synthesis inhibiting factor OsDNR1, and further inhibits auxin accumulation, root growth, and uptake of NO₃⁻. The OsRNR10 is knocked out in a japonica rice, the root system architecture of rice can be optimized, and the NUE and yield of the rice can be improved. The present invention further discovers that a difference of 3,496 bp generally exists in OsRNR10 promoter regions of indica rice and japonica rice. The specific difference is that a 3,496-bp sequence of −3,645th to −150th bp exists in a japonica rice OsRNR10 gene promoter, which is deleted in indica rice. In indica rice, the 3,496-bp deletion reduces the expression amount of the OsRNR10 gene, thereby enabling the indica rice to have a better root system architecture and nitrogen use efficiency. Therefore, the 3,496 bp deletion can be used as a marker for determining rice root system development and nitrogen use efficiency.

### BRIEF DESCRIPTION OF THE DRAWINGS

FIG. 1 shows root system architectures of different indica and japonica materials under different nitrogen culture conditions, where (a) indicates root system phenotypes of different indica and japonica subspecies under low-nitrogen and high-nitrogen culture conditions, (b) indicates a ratio of total root length, (c) indicates a ratio of total root area, and (d) indicates a ratio of total root tip number, and in the figure, HJX74 is an abbreviation of Huajingxian 74; TQ is an abbreviation of Teqing; ZF802 is an abbreviation of Zhefu 802; ZH11 is an abbreviation of Zhonghua 11; and WYJ7 is an abbreviation of Wuyunjing 7.

FIG. 2 shows differences in map-based cloning and sequences of a candidate gene OsRNR10 between HJX74 and IRAT261, where (a) indicates fine mapping of OsRNR10; and (b) indicates differences in OsRNR10 promoters, open reading frames, and protein coding regions between HJX74 and IRAT261.

FIG. 3 shows the nitrogen-induced expression of OsRNR10 and the activity analysis of OsRNR10 promoters, where (a) the transcription level and protein abundance of OsRNR10 increase along with the increase of an external nitrogen concentration, and (b) the activity of the HJX74 and IRAT261-derived OsRNR10 promoters is verified by using a transcriptional activation experiment.

FIG. 4 shows the root system phenotypes and NO₃⁻ uptake rate of OsRNR10 transgenic materials under the background of ZH11, where (a) indicates root system phenotypes of OsRNR10 transgenic materials under low-nitrogen and high-nitrogen culture conditions, (b) indicates a ratio of total root length, (c) indicates a ratio of total root area, (d) indicates a ratio of total root tip number, and (e) indicates the NO₃⁻ uptake rate.

FIG. 5 shows that protein interaction exists between OsRNR10 and OsDNR1 and the stability of OsDNR1 is affected, where (a) a split firefly luciferase complementation (SFLC) assay verifies that protein interaction between OsRNR10 and OsDNR1 exists in plants, (b) a rice protoplast system co-immunoprecipitation (Co-IP) assay verifies the protein interaction between OsRNR10 and OsDNR1, (c) indicates the transcription level of OsDNR1 in OsRNR10 transgenic materials, (d) indicates the protein abundance of OsDNR1 in the OsRNR10 transgenic materials, (e) indicates the degradation rate analysis of GST-OsDNR1 in OsRNR10-overexpressing materials, (f) indicates the degradation rate analysis of GST-OsDNR1 in osrnr10 materials, and (g) indicates the content of endogenous indole-3-acetic acid (IAA) in the OsRNR10 transgenic materials.

FIG. 6 shows that OsRNR10 mediates mono-ubiquitination modification of OsDNR1, where (a) OsRNR10 performs mono-ubiquitination modification of OsDNR1, and (b) indicates the degradation rate analysis of different mutant types of GST-OsDNR1 in OsRNR10-overexpressing lines.

FIG. 7 shows OsDNR1 expression amounts, root system phenotypes, and auxin content of NIL-OsDNR1^{HJX74}, NIL-OsDNR1^{IRAP9}, and NIL-OsDNR1^{IRAP9} osrnr10, where (a) indicates the transcription levels and protein abundance of OsDNR1, (b) indicates root system phenotypes under low-nitrogen and high-nitrogen culture conditions, (c) indicates a ratio of total root length, (d) indicates a ratio of total root area, (e) indicates a ratio of total root tip number, and (f) indicates the content of endogenous IAA.

FIG. 8 shows that indica-japonica differentiation exists in an OsRNR10 sequence, where (a) indicates the promoter region analysis of OsRNR10 of 12 indica rice and 12japonica rice, (b) indicates the open reading frame region analysis, (c) indicates the protein coding region analysis, (d) indicates the transcription level of OsRNR10, (e) indicates a phylogenetic tree of OsRNR10 in about 3,000 rice varieties, (f) indicates the haplotype analysis of OsRNR10, (g) indicates the nucleotide diversity analysis in a 20-kb region upstream and downstream of OsRNR10, and (h) indicates haplotype distribution overlapping of OsRNR10 and OsDNR1.

FIG. 9 shows root system changes and major agronomic traits of near isogenic lines NIL-OsRNR10^{HJX74} and NIL-OsRNR10^{IRAT261} under different nitrogen concentrations, where (a) indicates a ratio of total root length under low-nitrogen and high-nitrogen culture conditions, (b) indicates a ratio of total root area, (c) indicates a ratio of total root tip number, (d) indicates a plant height, (e) indicates tiller, (f) indicates a number of secondary branches, (g) indicates a number of grains, and (g) indicates yield per plant.

FIG. 10 shows the phenotypic comparison analysis of important agronomic traits of ZH11 and knockout lines osrnr10 under its background at different levels of nitrogen fertilisation, where (a) indicates a plant type, (b) indicates a plant height, (c) indicates tiller, (d) indicates a number of primary branches, (e) indicates a number of secondary branches, (f) indicates a number of grains per panicle, and (g) indicates yield per plant.

### DETAILED DESCRIPTION

In the following examples, HJX74 is an abbreviation of Huajingxian 74; and ZH11 is an abbreviation of Zhonghua 11.

### Example 1 Significant Differences are Confirmed to Exist in RSA Overall Size and Nitrogen-Dependent Plastic Development Response of Indica Rice and Japonica Rice

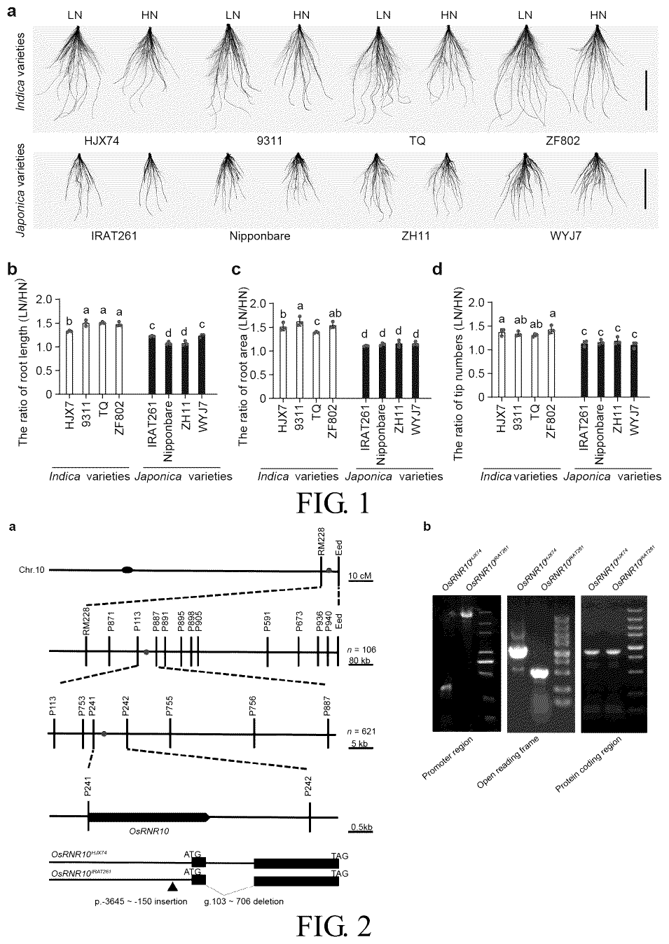

The inventors conducted water culture on 4 indica rice materials and 4 japonica rice materials in two nutrient solutions of high nitrogen (1.25 mM NH₄NO₃) and low nitrogen (0.375 mM NH₄NO₃) for 10 days respectively, then measured root system development indicators (root length, root area, and root tip number) of the different materials under low-nitrogen and high-nitrogen conditions, and calculated ratios of the root system development indicators under the low-nitrogen and high-nitrogen conditions. The result showed that compared with indica rice, japonica rice had the smaller RSA and weaker response to an external nitrogen response (FIG. 1).

### Example 2 Key Site OsRNR10 Causing Difference in Nitrogen Response of Indica Rice and Japonica Rice RSA is Identified

The inventors constructed 82 single segment substitution line materials (chromosome segment substitution lines, CSSLs) with IRAT261 as a donor parent and HJX74 as an acceptor parent, and the materials were subjected to water culture. It was found that compared with HJX74, SSSL-024 had the smaller RSA and weaker response to an external nitrogen response. SSSL-024 carried a fragment from IRAT261 on the short arm of chromosome 10. In order to finely map a nitrogen-response root system development regulatory factor, SSSL-024 backcrossed with HJX74 to construct 106 BC₁F₂ populations and 621 BC₂F₂ populations for fine mapping and map cloning, and a candidate gene was mapped to be within a range of 3.3 kb between markers P241 and P242. The promoter and genomic region of only one gene LOC_Os10g41838 existed in this region, and the gene was named OsRNR10 (REGULATOR OF N-RESPONSIVE RSA ON CHROMOSOME 10) (FIG. 2a). It was found through sequencing and sequence alignment that OsRNR10 of SSSL-024 had an additional 3,496-bp fragment (912nd-4,408th bp in SEQ ID NO: 5) in the promoter region, while OsRNR10 of HJX74 had a 604-bp insertion (103rd-707th bp in SEQ ID NO: 2) in the open reading frame region. But the fragment was spliced out during transcription, and therefore did not affect the protein coding sequence (FIG. 2b).

The molecular markers used in the research were PCR-based markers, including SSR markers and self-designed InDel markers. The SSR markers were all from the linkage map of microsatellite markers published by McCouch, et al. (2001, 2002); and the STS markers were SSR target sequences with good microsatellite repeatability screened by analyzing clone sequences using an SSR analysis tool (http://www.gramene.org/gramene/searches/ssrtool), and then a primer design was performed on these target sequences using Primer5 analysis software. The sequences of polymorphic marker primers used for fine mapping and map cloning were detailed in Table 1.

TABLE 1

| Primers | Forward sequence (5′-3′) | Reverse sequence (5′-3′) |
|---|---|---|
| RM228 | CTGGCCATTAGTCCTTGG | GCTTGCGGCTCTGCTTAC |
| P871 | GAAGTCCGAACGGCAGAAACC | CGATTCTACGGACGGGTAGGC |
| P113 | GACATGCGAACAACGACATC | GCTGCGGCGCTGTTATAC |
| P887 | GCGGAGCAACCAAGCCTACC | ATCGGAGGAAGCAAACCAAACG |
| P891 | TGTCTACGGCTGCCGAAACTCTCC | ATGGGCCTAGCAGCAGCAGAAG |
| | | C |
| P895 | CTCTGTCGTCCACTCCAGATTCC | GTCGAATTGAGAGGGATTGATG |
| | | G |
| P898 | GCAGCCAATCAGAAATCATAGC | AAATATGTCCGTGTCCCAATGC |
| P905 | CGCCCAAATAAATGCTGCAGACC | GCGTGAACTAACCACACCCGTA |
| | | GC |
| P591 | CTCATAGGTGGGTTAGTTTCTTGG | GCTGGTTTACAACTTGCTACTCT |
| | | ACC |
| P673 | CCACCCGTCTCATGTTCTACTCG | ATGGAGATGATACACTCGCATCG |
| P936 | CTGGTTAGCCTCCGATCCTTCG | ACGTAACGCTTCTCAAGCCACA |
| | | CC |
| P940 | CGGTGCCTTCACCACACATCG | GAAAGCAAAGGGAGTGAGGGA |
| | | GATGG |
| P940 | CGGTGCCTTCACCACACATCG | GAAAGCAAAGGGAGTGAGGGA |
| | | GATGG |
| P753 | AAAAGCAGCTGTGATCCTTCA | GTGCGGAAGGAAATTCAAGA |
| P241 | TGCTACGCTTCAAGTCCGTC | GAGCTTGGTGATAGTCGGGG |
| P242 | AATTGGACCGAGTGTTGTT | GCGAAGAATTAGCCATTAGC |
| P755 | CATTTGAGGTGCATTGGATG | TAGGCGTATGTGCTGGTTGA |
| P756 | ATACTCACAAGCGACTGGGC | AATCACCAAACACATGGCTG |
| OsRNR10- | CTTACCACCATATCTGAATCAC | GCTGATGAGGATGGATGG |
| Promoter | (SEQ ID NO: 9) | (SEQ ID NO: 10) |
| OsRNR10- | CCCACTGTGATGGCCTAGTG | ATAAGCACCCCAGCCTTGTC |
| gDNA | | |
| OsRNR10- | ATGGATGCCAAGGACTTCCC | CTAAGCAGCGGTAATTCTAA |
| ORF | | |
| OsRNR10- | ACTCACTATAGGGCGAATTGGGTAC | ATATCAAGCTTATCGATACCGTC |
| G0800 | CGTCAAAGTGATCTTATTCCT | GACGATCCTCGCAAGGGAGGAG |
| | | G |
| OsRNR10- | GGGGACAAGTTTGTACAAAAAAGC | ggggaccactttgtacaagaaagctgggtcAGC |
| Getway | AGGCTCCATGGATGCCAAGGACTT | AGCGGTAATTCTAA |
| | CCC | |

The PCR procedure was carried out according to the method of Panaud, et al. (1996) with a slight modification. Specifically, 20 μL per tube of an amplification reaction system included: 0.15 μM SSR primers, 200 μM dNTPs, 1×PCR reaction buffer (50 mM KCl, 10 mM Tris-HCl pH 8.3, 1.5 mM MgCl₂, 0.01% gelatin), 50-100 ng template DNA, and 1 U Taq enzyme. The reaction procedure was as follows: DNA denaturation at 94° C. for 5 min, cycling (94° C. for 1 min, 56° C. for 1 min, and 72° C. for 1 min) for 36 times, and re-extension at 72° C. for 5 min. The amplified PCR product was subjected to electrophoresis using 6% polyacrylamide denatured gel, and gel imaging was performed after the electrophoresis.

### Example 3 Expression of OsRNR10 Increases with Increase of Nitrogen Concentration, and Promoter Activity of OsRNR10^{IRAT261} is Higher than that of OsRNR10^{HJX74}

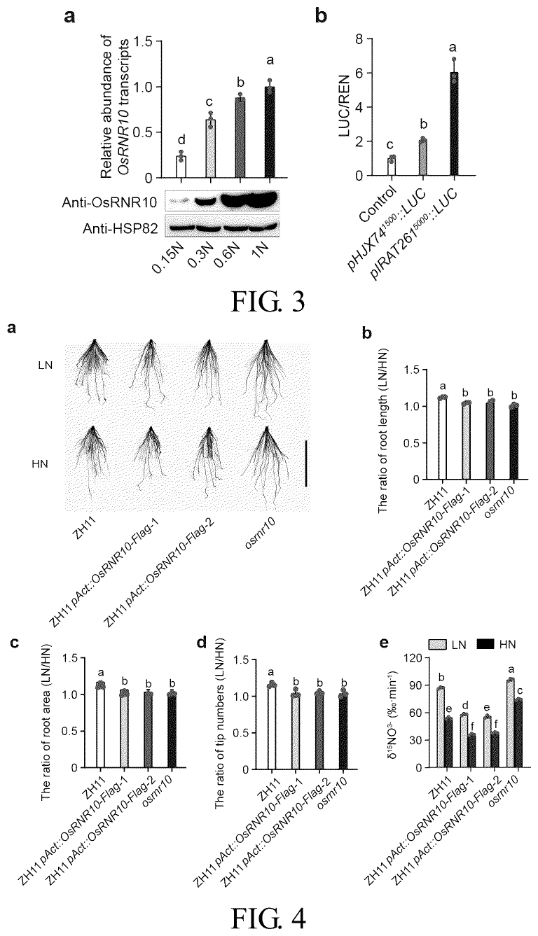

The inventors performed a water-culture experiment on HJX74 using nutrient solutions of four nitrogen concentrations (0.15 N, 0.1875 mM NH₄NO₃; 0.3 N, 0.375 mM NH₄NO₃; 0.6 N, 0.75 mM NH₄NO₃; and 1 N, 1.25 mM NH₄NO₃). Total RNA was extracted from the root tip of HJX74 water-cultured for 4 weeks, and the transcription level and protein level of OsRNR10 were analyzed by qRT-PCR and Western-blotting experiments. The result showed that the expression amount of OsRNR10 increased with the increase of nitrogen concentration (FIG. 3a). In addition, the inventors also found through a protoplast transient expression analysis experiment that the promoter activity of OsRNR10 from IRAT261 was higher than that of OsRNR10 from HJX74 (FIG. 3b).

The experimental method for transcriptional activation used in the example was briefly described as follows: OsRNR10 promoter fragments of about 1.5 kb and 5 kb (the primer sequence was shown in OsRNR10-G0800 of Table 1) were respectively amplified from HJX74 and IRAT261, and the fragments were inserted into an upstream of a LUC gene in a pGreenII 0800-LUC vector to construct a pHJX74¹⁵⁰⁰::LUC vector and a pIRAT261⁵⁰⁰⁰::LUC vector. The coleoptiles of rice seedlings were cut into strips, placed in enzymolysis liquid, and subjected to lysis under low-speed shaking in a dark place for 5 hours. An equal volume of a W5 solution (154 mM NaCl, 125 mM CaCl₂, 5 mM KCl, 2 mM MES, pH 5.7) was added. The solution was subjected to a 200-g low-speed centrifugation for 5 minutes, and a precipitate was collected, resuspended and washed with W5, repeatedly washed 2 times, and then resuspended with an MMg solution (0.4 M mannitol, 15 mM MgCl₂, 4 mM MES, pH 5.7). pGreenII 0800-LUC, pHJX74¹⁵⁰⁰::LUC and pIRAT261⁵⁰⁰⁰::LUC vectors were added to rice protoplasts. Transformation was performed with an equal volume of PEG4000/Ca²⁺. After 15 minutes of standing, 2 volumes of W5 were added to terminate the reaction, and the product was washed, placed in the W5 solution, and dark-cultured for 16 hours. A lysate was added after the protoplasts were collected, and the LUC/REN value was determined using a PROMEGA dual-fluorescence detection kit.

### Example 4 Knockout Line and Overexpressing Line Under Background of Japonica Rice Variety ZH11 are Constructed, and Regulation and Control of Rice RSA and NO₃⁻ Uptake Rate by OsRNR10 are Confirmed

In order to further clarify the function of OsRNR10, the inventors constructed an OsRNR10 knockout vector by using a CRISPR/Cas9 knockout system. Meanwhile, an OsRNR10-overexpressing vector pAct::OsRNR10-Flag was constructed. The two vectors were introduced into the japonica rice variety ZH11 by using an agrobacterium-mediated transformation method, and an OsRNR10 knockout line osrnr10 and an overexpressing line ZH11/pAct::OsRNR10-Flag were constructed. Subsequently, the root system development indicators of the ZH11/pAct::OsRNR10-Flag and osrnr10 were measured using the high and low-nitrogen water culture systems of example 1. It was found that the ZH11/pAct::OsRNR10-Flag and osrnr10 showed less sensitive RSA reaction to the external nitrogen fertilisation amount compared to wild-type ZH11. Besides, the ZH11/pAct::OsRNR10-Flag showed smaller RSA and the osrnr10 showed larger RSA (FIGS. 4a-d). Meanwhile, the NO₃⁻ uptake rate was measured. The result showed that compared with the wild-type ZH11, the NO₃⁻ uptake rate of the ZH11/pAct::OsRNR10-Flag was significantly reduced, while the NO₃⁻ uptake rate of the osrnr10 was significantly increased (FIG. 4e).

The NO₃⁻ uptake rate measurement method used in the example was as follows: rice seeds were sterilized with a 20% sodium hypochlorite solution for 30 minutes. Then the seeds were placed in an incubator at 37° C. and soaked in water to be imbibed for 24 hours. The seeds were drained and transferred to an incubator at 28° C. for accelerating germination. After white buds showed, the seeds were transferred to a hollow 96-well plate to be cultured for 7 days. Seedlings with consistent growth vigor were selected and transferred to a blue box containing 40 L of a nutrient solution (1.25 mM NH₄NO₃, 0.5 mM NaH₂PO₄·2H₂0, 0.75 mM K₂SO₄, 1 mM CaCl₂, 1.667 mM MgSO₄·7H₂O, 40 μM Fe-EDTA (Na), 19 μM H₃BO₃, 9.1 μM MnSO₄·H₂O, 0.15 μM ZnSO₄·7H₂O, 0.16 μM CuSO₄, and 0.52 μM (NH₄)₃Mo₇O₂₄·4H₂O, pH 5.5). For treatments with different nitrogen concentrations, standard nutrients, 0.3 N (0.375 mM NH₄NO₃), and 0.15 N (0.1875 mM NH₄NO₃) were used, the seeds were cultured for 3 weeks, and the pH value was adjusted once a day.

After the 3 weeks of culture, rice roots were soaked in 0.1 mM CaSO₄ for 1 minute, transferred to a nutrient solution containing 2.5 mM K¹⁵NO₃ for 5 minutes, and finally transferred to 0.1 mM CaSO₄ for 1 minute. The water of the root system was sucked dry by using filter paper or gauze. The root system was cut off, dried, and ground. Then the ¹⁵N content was measured (finished by the Li Yuzhong laboratory of Chinese Academy of Agricultural Sciences, and the used instrument was Isopirme 100).

### Example 5 OsRNR10 Positively Regulates and Controls Stability of Auxin Synthesis Inhibiting Factor OsDNR1 so as to Reduce Rice Auxin Accumulation

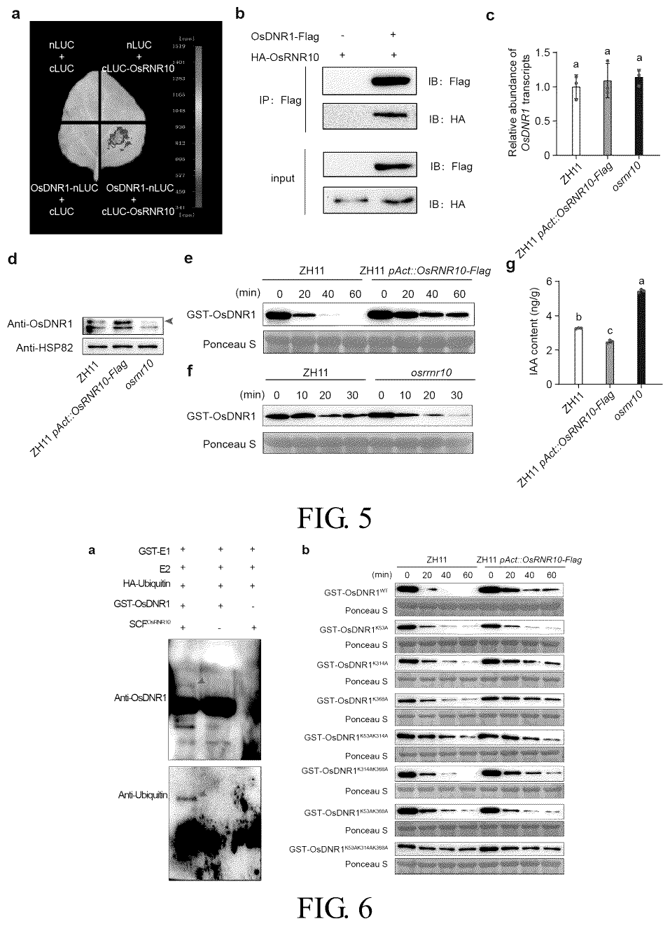

The inventors predicted that OsRNR10 belongs to the FBA protein family to which the F-box family belongs by a protein domain analysis, and might function as the F-box protein. Therefore, to determine downstream targets of OsRNR10, co-immunoprecipitation combined with mass spectrometry (IP-MS) was conducted and 4 high-quality peptide fragments from OsDNR1 were identified. Furthermore, the interaction of OsRNR10 with OsDNR1 was confirmed by a split firefly luciferase complementation (SFLC) assay and a rice protoplast co-immunoprecipitation (Co-IP) assay (FIGS. 5a-b). Subsequently, the OsDNR1 transcription level and protein abundance in ZH11/pAct::OsRNR10-Flag and osrnr10 were detected. The result showed that although no difference was detected in the transcription level of OsDNR1, the protein abundance of OsDNR1 was significantly increased in the ZH11/pAct::OsRNR10-Flag, but significantly decreased in osrnr10 (FIGS. 5c-d), indicating that OsRNR10 can regulate and control the protein abundance of OsDNR1 at the protein level.

On this basis, a cell-free degradation assay was used to detect the degradation rates of GST-OsDNR1 (glutathione mercaptotransferase) in the wild-type ZH11, ZH11/pAct::OsRNR10-Flag, and osrnr10. The result showed that compared to the wild type, the degradation rate of the GST-OsDNR1 was decreased in the ZH11/pAct::OsRNR10-Flag and increased in the osrnr10 (FIG. 5). Meanwhile, the content of auxin of the OsRNR10 transgenic materials was determined. The result showed that the IAA level was decreased in the ZH11/pAct::OsRNR10-Flag, and more IAA was accumulated in the osrnr10. Therefore, it was speculated that OsRNR10 positively regulated and controlled the stability of an auxin synthesis inhibiting factor OsDNR1 so as to reduce the rice auxin accumulation.

The SFLC assay method used in the example was briefly described as follows: full-length OsRNR10 and OsDNR1 cDNAs were amplified (using ZH11 as a template, and primers shown in OsRNR10-Getway of Table 1), and then inserted into pCAMBIA1300-35SCluc-RBS and pCAMBIA3300-35S-HA-Nluc-RB respectively (Chen, H. et al., Firefly Luciferase Complementation Imaging Assay For Protein-protein Interactions in Plants. Plant Physiol. 146, 368-376 (2008). cLUC-OsRNR10, OsDNR1-Nluc, and silencing plasmid p19 were transfected into tobacco epidermal cells simultaneously by agrobacterium-mediated transformation. After 48 h incubation, the injected tobacco leaves were coated with 1 mM fluorescein (Promega, E1605) and the LUC signal was observed with LB985 (Berthold). For vectors and specific assay methods, reference was made to Chen, et al. (2008).

The Co-IP assay method used in the example was briefly described as follows: the full-length OsRNR10 and OsDNR1 cDNAs were amplified (using ZH11 as a template and primers shown in OsRNR10-Getway of Table 1), and then inserted into pUC-35S-Flag-RBS and pUC-35AS-HA-RBS vectors respectively (Liu Q, et al., G-protein βγ Subunits Determine Grain Size Through Interaction with MADS-domain Transcription Factors in Rice, Nat. Commun. 9, 852 (2018). The rice protoplasts were transfected with 100 μg of plasmids (the rice protoplasts were prepared as described in example 3), and the protoplasts were cultured under low light for 18 h, followed by 50 mM HEPES (pH 7.5), 150 mM KCl, 1 mM EDTA (pH 8), 0.3% Trition-X 100, 1 mM DTT, and an added protease inhibitor (Roche Life Science). Lysates were incubated with magnetic beads conjugated with a DDDDK-tagged antibody (MBL, M185-11) respectively at 4° C. for 4 hours. The magnetic beads were then washed 6 times with a lysis buffer and eluted with 3×Flag peptide (Sigma-Aldrich, F4709). Immunoprecipitates were separated by an SDS-PAGE electrophoresis, and target proteins were detected by immunoblotting with DDDDK (MBL, M185-7) or HA (MBL, M180-7) antibodies. The immunoblotting result was visualized on a Tanon-5200 chemiluminescence imaging system (Tanon Science and Technology). For vectors and specific assay methods, reference was made to Liu, et al. (2018).

The cell-free degradation assay method used in the example was briefly described as follows: rice seedlings grown for 14 days were harvested and ground with liquid nitrogen, and a cell lysate was extracted with 25 mM Tris-HCl (pH 7.5), 10 mM NaCl, 10 mM MgCl₂, 4 mM PMSF, 5 mM DTT, and 10 mM ATP. 200 μL of the rice cell lysate was incubated with 100 ng of a purified GST-OsDNR1 fusion protein for a series of time. The proteins were extracted and then separated by the SDS-PAGE electrophoresis. The target proteins were detected by immunoblotting with an anti-GST antibody (MBL, PM013-7). Ponceau S stained blots were used as a loading control.

### Example 6 OsRNR10 Inhibits Ubiquitination Degradation of OsDNR1 by Monoubiquitinating K53 Site of OsDNR1

Through mass spectrometry, the inventors revealed that three lysine sites (K53, K314, and K368) of OsDNR1 were ubiquitinated in plants. Since OsRNR10 may function as a F-box protein to form an SCF complex in plants to play a function of an E3 ubiquitin ligase for ubiquitination modification of a substrate, an in-vitro ubiquitination experiment was performed. The result showed that in the presence of a ubiquitin activating enzyme E1, a ubiquitin conjugating enzyme E2, and ubiquitin, GST-OsDNR1 could be subjected to mono-ubiquitination modification by an OsRNR10-Flag fusion protein (FIG. 6a). To further reveal the site of OsRNR10-monoubiquitinated OsDNR1, the degradation rates of the mutant fusion protein with one or more lysine sites of GST-OsDNR1 substituted by alanine in cell-free degradation systems of ZH11 and ZH11/pAct::OsRNR10-Flag. The result showed that compared with wild-type GST-OsDNR1, the GST-OsDNR1 mutant in which the lysine at K53 site was substituted with phenylalanine was no longer stable in the ZH11/pAct::OsRNR10-Flag, whereas the change of the mutation of the lysine at two other sites still improved the stability of the GST-OsDNR1 in the ZH11/pAct::OsRNR10-Flag (FIG. 6b). Therefore, it was concluded that OsRNR10 inhibited ubiquitination degradation of OsDNR1 by mono-ubiquitinating the K53 site of the OsDNR1.

The in-vitro ubiquitination assay method used in the example was briefly described as follows: the OsRNR10 fusion protein was immunoprecipitated from a protein extract of ZH11 pAct::OsRNR10-Flag using the magnetic beads conjugated with the DDDDK-tagged antibody (MBL, M185-11), and then eluted with 3×Flag peptide (Sigma-Aldrich). The purified OsRNR10-Flag protein was used for the in-vitro ubiquitination assay. GST-OsDNR1, HA-Ubiquitin (U-110-01M, R&D), GST-E1 (UBE1, E-306-050, R&E), E2 (E2-607-100, R&D), and purified E3 (RNR10-Flag) were used for the protein ubiquitination analysis, and the buffer contained 50 mM Tris-HCl (pH 7.4), 5 mM MgCl₂, and 2 mM ATP. The reactant was incubated at 30° C. for 5 hours. The product was separated by the SDS-PAGE electrophoresis. The protein was detected by immunoblotting using an OsDNR1 antibody (ABclonal) and a Ubiquitin antibody (abeam, ab134953).

### Example 7 OsRNR10 Acts Upstream of OsDNR1 and Regulates Auxin Homeostasis and Root System Phenotype

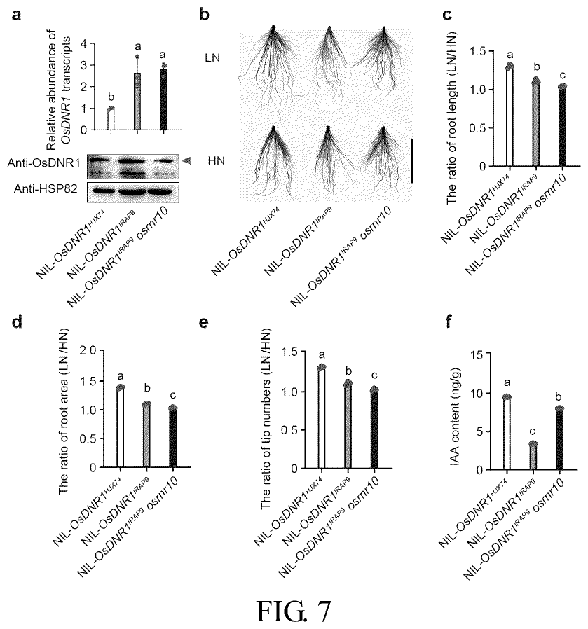

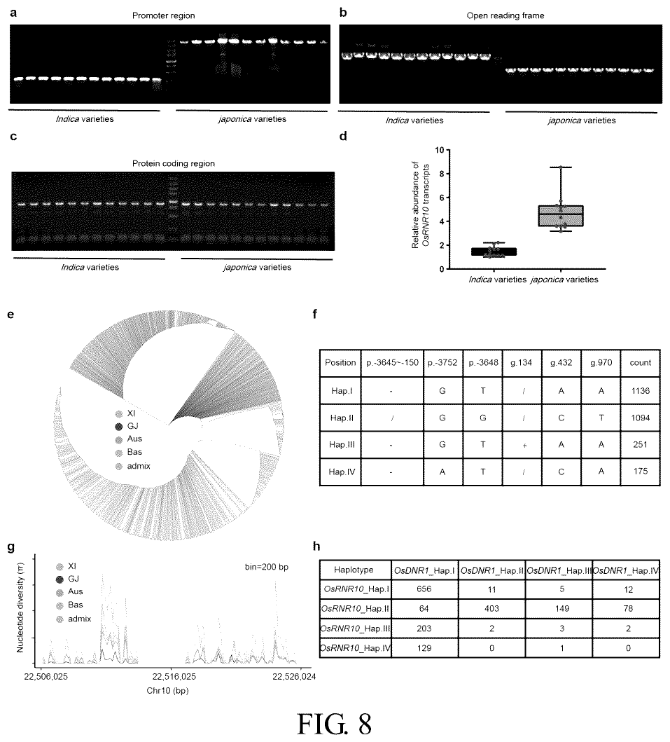

The inventors constructed an OsRNR10 knockout line NIL-OsDNR1^{IRAP9} osrnr10 using a CRISPR/Cas9 knockout system under the background of an OsDNR1 near isogene NIL-OsDNR1^{IRAP9} line constructed in the early stage. Then the transcription level and protein abundance of the OsDNR1 in NIL-OsDNR1^{HJX74}, NIL-OsDNR1^{IRAP9}, and NIL-OsDNR1^{IRAP} osrnr10 were detected. The result showed that the transcription level of the OsDNR1 was not changed in the NIL-OsDNR1^{IRAP} osrnr10, but its protein abundance was significantly decreased (FIG. 7a). The root system phenotype analysis also showed the NIL-OsDNR1^{IRAP9} osrnr10 root system had a further decrease in nitrogen sensitivity (FIGS. 7b-e), and the endogenous auxin level was also increased (FIG. 7f). Taken together, it was concluded that the OsRNR10 acted upstream of the OsDNR1 and regulated auxin homeostasis and root system phenotype of rice.

### Example 8 Indica-Japonica Differentiation Existing in OsRNR10 Causes Response Differences of RAS of Indica Rice and Japonica Rice to External Nitrogen

Compared with a promoter (SEQ ID NO: 1) of an OsRNR10^{HJX74} gene, the difference of a 3,496-bp inserted fragment and 25 SNPs exists in a promoter (SEQ ID NO: 5) of an allele OsRNR10^{IRAT261}, while the OsRNR10^{HJX74} had a 603-bp insertion in the open reading frame region (SEQ ID NO: 2). The inventors analyzed the OsRNR10 gene in 12 indica rice materials and 12 japonica rice materials (shown in Table 2). It was found that all the indica rice varieties had a 3,496-bp deletion of the promoter region and a 604-bp insertion of the open reading frame region. Besides, the 604-bp insertion of the open reading frame region was spliced out during transcription, without affecting the encoded amino acid sequence (FIGS. 8a-c). Meanwhile, the transcription levels of OsRNR10 were detected in 24 rice varieties. It was found that the transcription level in the japonica rice varieties was significantly higher than that in the indica rice varieties (FIG. 8d). Therefore, the 3,496-bp deletion of the promoter region of the OsRNR10 of the indica rice varieties resulted in the low expression amount of the OsRNR10, which enables the indica rice to have larger overall RSA and stronger response to external nitrogen, and also have a higher NO₃⁻ uptake rate.

The inventors further performed a phylogenetic analysis using about 3,000 rice germplasms. The result showed significant differentiation of the OsRNR10 in indica rice and japonica rice (FIG. 8e). The haplotype analysis of these varieties showed 4 haplotypes of the OsRNR10, with the majority of the rice varieties Hap.I and Hap.II (83.9%). Besides, 98.7% of the japonica rice was Hap.II and 69.1% of the indica rice was Hap.I, showing a strong selection preference of indica and japonica subspecies (FIG. 8f). Meanwhile, the nucleotide polymorphism analysis of 20-kb upstream and downstream of the OsRNR10 also proved that the directed selection drove the differentiation of the OsRNR10 in the indica rice and japonica rice (FIG. 8g). The haplotype distribution pattern analysis of the OsRNR10 and OsDNR1 indicated that there was a large overlap between the two genes, and the two genes synergistically regulated and controlled the RAS, nitrogen responsiveness, and NO₃⁻ uptake rate of the rice (FIG. 8h).

TABLE 2

| Name of rice variety | Subspecies |
|---|---|
| Xiangaizao No. 4 | Indica rice |
| Guangluai No. 4 | Indica rice |
| Minghui 63 | Indica rice |
| Wenxuanqing | Indica rice |
| Yuanfengzao | Indica rice |
| Xiangaizao No. 9 | Indica rice |
| Qingxuanzao | Indica rice |
| Zhefu 802 | Indica rice |
| Teqing | Indica rice |
| 9311 | Indica rice |
| Nanjing No. 6 | Indica rice |
| Huajingxian 74 | Indica rice |
| 313 | Japonica rice |
| 314 | Japonica rice |
| Zhonghua 11 | Japonica rice |
| Wuyunjing No. 7 | Japonica rice |
| Taizhong 65 | Japonica rice |
| Xiushui 09 | Japonica rice |
| Xiushui 110 | Japonica rice |
| Nipponbare | Japonica rice |
| Longdao No. 5 | Japonica rice |
| Lansheng | Japonica rice |
| Qianchonglang No. 2 | Japonica rice |

### Example 9 Near Isogenic Lines NIL-OsRNR10^{HJX74} and NIL-OsRNR10^{IRAT261} Under the Background of Indica Rice Variety HJX74 are Constructed, and Comparative Analysis of Agronomic Traits Such as Yield is Completed

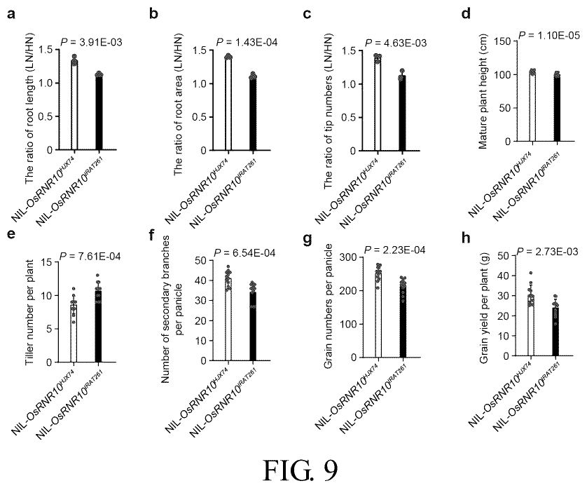

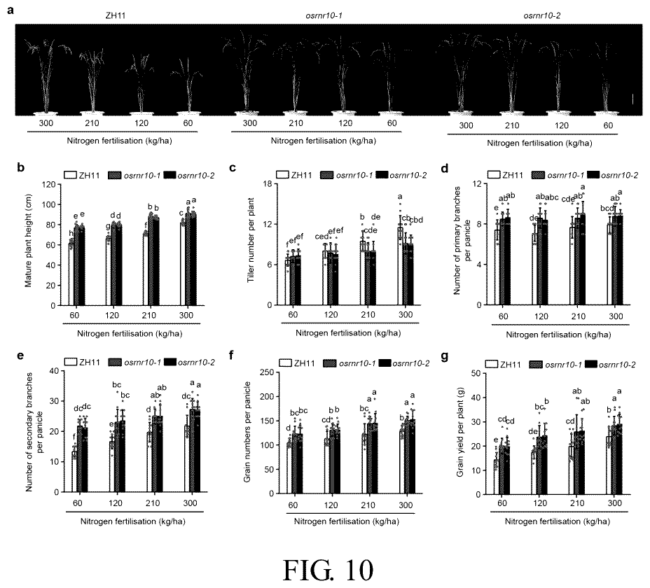

The inventors selected a material carrying an OsRNR10^{IRAT261} site from the BC₂F₂ population described in example 2 to be continuously backcrossed with HJX74 for three times, and finally obtained a pair of near isogenic lines NIL-OsRNR10^{HJX74} and NIL-OsRNR10^{IRAT261} under the background of HJX74. The near isogenic lines were water-cultured under low-nitrogen and high-nitrogen conditions, and the root system development indicators were measured. The result showed that compared with the NIL-OsRNR10^{HJX74}, the NIL-OsRNR10^{IRAT261} had smaller RSA and a weak response to external nitrogen (FIGS. 9a-c). A predicted yield test was performed on the field with normal nitrogen fertilization amount (210 kg/ha), and various important agronomic traits were observed and counted. The comparative statistical result showed that the allelic variation OsRNR10^{IRAT261} in japonica rice increased the number of tillers of rice, but significantly reduced the plant height, the number of primary branches, the number of secondary branches, and the number of grains per panicle, and finally decreased the yield per plant (FIGS. 9d-h).

The specific statistical method was as follows: the plant height was counted: after the rice was ripe, 16 plants were taken out in the field respectively to measure the plant height. Tillers were counted: after the rice was ripe, 12 plants were taken out in the field respectively to measure the number of tillers. The number of grains was counted: after the rice was ripe, 12 panicles on main tillers were taken out in the field respectively to respectively directly count the number of grains per panicle and record the number. The yield per plant was counted: after the rice was completely ripe, 12 single plants in the plot were threshed, the harvested seeds were dried at a constant temperature of 37° C. and weighed to obtain the data of yield per plant, and 3 times of repeated tests needed to be performed.

### Example 10 Agronomic Traits Such as Yield of OsRNR10 Knockout Line Under the Background of Japonica Rice ZH11 Under Different Nitrogen Fertilisation Levels

The OsRNR10 knockout line osrnr10 constructed by the inventors in example 4 was used for a field trial. The wild-type ZH11 and the knockout line osrnr10 were planted in the fields with different nitrogen fertilization amounts (60 kg/ha, 120 kg/ha, 210 kg/ha, and 300 kg/ha, respectively), and the agronomic traits of the knockout line and the wild type were observed and counted. The result showed that when the nitrogen fertilisation amount was the same, osrnr10 can slightly increase the plant height of rice, reduce the number of tillers, and significantly increase the number of secondary branches and the number of grains. Therefore, compared with the ZH11, the osrnr10 had higher NUE and yield per plant. In addition, under the condition of low nitrogen fertilisation, the yield improvement caused by the osrnr10 was more significant (FIG. 10), which showed that the decrease of the expression of the OsRNR10 can ensure that the rice can also keep relatively better yield under the condition of low nitrogen fertilisation level, and the fertilisation amount of the nitrogen fertilizer was saved.

1. A method comprising using an indica rice OsRNR10 gene promoter in optimizing rice root system development, and improving rice nitrogen use efficiency and yield, wherein a sequence of an indica rice OsRNR10 gene promoter is shown in SEQ ID NO: 1.

2. The method of claim 1, further comprising using an indica rice OsRNR10 gene in optimizing rice root system development, and improving rice nitrogen use efficiency and yield wherein a gDNA nucleotide sequence is shown in SEQ ID NO: 2.

3. A method comprising detecting whether a marker related to nitrogen-dependent regulation and control of rice root system development and rice nitrogen use efficiency exists or is deleted, wherein the marker is a 3,496-bp sequence of 912nd-4,408th bp in an OsRNR10 gene promoter shown in SEQ ID NO: 5; the marker exists in japonica rice, causes a high expression of OsRNR10, weakens root system development, and enables a root system to be insensitive to an external nitrogen source change and have a slow uptake rate of nitrate nitrogen; and the marker is deleted in indica rice, causes a low expression of the OsRNR10, and enables the root system to be more developed and more sensitive to the external nitrogen source change, and have an increased uptake rate of the nitrate nitrogen.

4. A method for determining rice root system development and nitrogen use efficiency, comprising performing the method of claim 3, wherein, if the marker exists, the rice variety root system development is relatively weak, and the nitrogen use efficiency is low; and if the marker is deleted, the rice variety root system development is relatively strong, and the nitrogen use efficiency is high.

5. The method according to claim 3, wherein sequences of primers in detecting the rice OsRNR10 gene promoter are shown in SEQ ID NO: 9 and SEQ ID NO: 10.

6. A method for enhancing rice root system development and nitrogen use efficiency, wherein an OsRNR10 gene in rice is knocked out or silenced.


SEQUENCE LISTING (10 sequences)

SEQ ID NO: 1 - DNA, 1045 bp, Oryza sativa
  source 1..1045: mol_type = genomic DNA
cttttatcttctcccaatgcataaagcaataacaaacatattactttattccttaatatcatatttgcatgcacattgtggatgccctaacatgaagtatgaaattatattggttttaggggcatagaagtttacataagatgcattcttctagtatgtaagaagaaacttacaactcttcatacatgcatatattccaccatgcatcacatcaggtcaggcatatacttcctccgtttcacaatgtaagaatttctagcattacctacatttatatagatgttatgtgaaacggagggagtagctaataaggcattccttaccaccatatctgaatcactaccgccgttgccgttgtataggctgtatgttcatctgtttgtttggttttccctttcacttgtttaattcattcttagcacaaagaaccaacaaaaaaaataagaagctctcttatcattgtcctgttggatatatcatctcagaatttgacgcagatgagatcacagctagtctatttgcatatagcccaagaagcagacagaacaatctttttcctgctcctcgaattagctgtgctccagaatccgatttttttttttggtttttggtatgcagattccgattcggactatgctatgttacgttccgttaataaatatatgtgcgctatgcaatctcgagttaagcatcatttgtttgtgtgtttgctgcatgtaggacgacaccatgaacatgaagaagaggagcaagaagaggaagaggacggtaggaccagctgcagcagcagctacttttcctgagctgccggaggagattgtgatggagattctggctcgtctgcccgtcaaatccctgctacgcttcaagtccgtctgcagaggttggcgtgcaatcatctccgagccctccttcatccgaacccaactccagtgctcggcttccagacggcagcaggagccatccatcctcatcagcccccaaattctccacccacatcagcttctaccagtgggagcaaggtgcctcctcccttgcgaggatc

SEQ ID NO: 2 - DNA, 1489 bp, Oryza sativa
  source 1..1489: mol_type = genomic DNA
atggatgccaaggacttccctgtcggccacaagttccgcttaatcagccactacacccactgtgatggcctagtgctcgccccgactatcaccaagctctacggggctgtttggttgccaccctgagacaaatatgcctggccggagataagcccgagaaaaaatgcatcttgtttggttggtgcctgaggcgctccatgccttccctggcctgagctctccggcagctcaatcagcctgagtcaggcgaccaaatttggtggcctgagttgcatgcagtgccaggcctcttgattagctgaagtgcttatgtactaattgttgaattaaagtaattctcttacgctgcagttggttgctttttttgcatgctgattggaaaacatgtcaaactactctccacttgattagctttgactttttctacaaatttggtaaacttaaaaggtttgactaaaaaaacaaaacgacttataatatgaaacagaaatagtactattaaactccaagcttaaaaagagaaagatactacataatattaaaactacataatattagttttaagtgttctcaggcacaggtcaccaaccaaacaggtttctctcaggcatgcctctcaggcatgaaccactcccacctcatgcaacactcagacacagatttttctcaggcatggtgctcaggtcaccaaccaaacaggccctacctcttcaacccggcaacaagagaagccatcacgttgcctgacggccatggccacagccacaccgcagggctggggctggatcctggtaccggcaggtacaaggtggttcggtccttctaccgctccccctccatggatcctcccgtctccatggggatggaggtgctcaccgtcggcgagcccggcgcgcggtggagggagaccgccgttgatcctccacaccccatcacccgatggcggaccgcccttgccgtcaatggtggctacctgttctggtacatggacaggcgccgctaccacgacgatgctccccggggactcctccggttcagcctgcgagacgaggcgttcgccgtaacccgtctcccggaatcgatggacccgacgctcgacgagaacgtcttgcccgacgtgctgcacggggagctgtgcgtcgtgcaagctctgcccgacaaggctggggtgcttatctggacaatgtcgtcgtcgtccatggacaacgacgatgtccacctcgacgacgggccatgggaactgcgatattgcatctgtgtcaacgccctttgccatccgttgggggttcttcctgatggaggaggaatcttgttgtgggcgaatcgcagcgtccatcgctacgacttctcggctaggaagctcgctggtgtggtgtgtaacctggatcgcatcaggtatcagggtggtaggccagcgagatggaagagtgtcgtcgacttcaccttgatgccctacaccgagtctctcgttagaattaccgctgcttag

SEQ ID NO: 3 - DNA, 885 bp, Oryza sativa
  source 1..885: mol_type = genomic DNA
atggatgccaaggacttccctgtcggccacaagttccgcttaatcagccactacacccactgtgatggcctagtgctcgccccgactatcaccaagctctacctcttcaacccggcaacaagagaagccatcacgttgcctgacggccatggccacagccacaccgcagggctggggctggatcctggtaccggcaggtacaaggtggttcggtccttctaccgctccccctccatggatcctcccgtctccatggggatggaggtgctcaccgtcggcgagcccggcgcgcggtggagggagaccgccgttgatcctccacaccccatcacccgatggcggaccgcccttgccgtcaatggtggctacctgttctggtacatggacaggcgccgctaccccgacgatgctccccggggactcctccggttcagcctgcgagacgaggcgttcgccgtaacccgtctcccggaatcgatggacccgacgctcgacgagaacgtcttgcccgacgtgctgcacggggagctgtgcgtcgtgcaagctctgcccgacaaggctggggtgcttatctggacaatgtcgtcgtcgtccatggacaacgacgatgtccacctcgacgacgggccatgggaactgcgatattgcatctgtgtcaacgccctttgccatccgttgggggttcttcctgatggaggaggaatcttgttgtgggcgaatcgcagcgtccatcgctacgacttctcggctaggaagctcgctggtgtggtgtgtaacctggatcgcatcaggtatcagggtggtaggccagcgagatggaagagtgtcgtcgacttcaccttgatgccctacaccgagtctctcgttagaattaccgctgcttag

SEQ ID NO: 4 - protein, 294 aa, Oryza sativa
MDAKDFPVGHKFRLISHYTHCDGLVLAPTITKLYLFNPATREAITLPDGHGHSHTAGLGLDPGTGRYKVVRSFYRSPSMDPPVSMGMEVLTVGEPGARWRETAVDPPHPITRWRTALAVNGGYLFWYMDRRRYPDDAPRGLLRFSLRDEAFAVTRLPESMDPTLDENVLPDVLHGELCVVQALPDKAGVLIWTMSSSSMDNDDVHLDDGPWELRYCICVNALCHPLGVLPDGGGILLWANRSVHRYDFSARKLAGVVCNLDRIRYQGGRPARWKSVVDFTLMPYTESLVRITAA

SEQ ID NO: 5 - DNA, 4556 bp, Oryza sativa
  source 1..4556: mol_type = genomic DNA
cttttatcttctcccaatgcataaagcaataacaaacatattactttattccttaatatcatatttgcatgcacattgtggatgccctaacatggagtatgaaattatattccttttaggggcatagaagtttacataagatgcattcttctagtacgtaagaagaaacttacaactcttcatacatgcatatattccaccatgcatcacatcaggccaggcatatacttcctccgtttcacaatgtaagaatttctagcattacctacatttatatagatgttatgtgaaacggagggagtagctaataaggcattccttaccaccatatctgaatcactaccgccgttgccgttgtataggctgtatgttcatctgtttgtttggttttccctttcacttgtttaattcattcttagcacaaagaaccaacaaaaaaaaataagaagctctcttatcattgtcctgttggatatatcatctcagaatttgacgcagatgagatcacagctagtctatttgcatatagcccaagaagcagacagaacaatctttttcctgctcctcgaattagctgtgctccagaatccgattcttttttttgtttgtttttggtatgcagattccgattcggactatgctatgttacgttccgttaataaatatatgtgcactatgcaatctcgagttaagcatcatttgtttgtgtgtttgctgcatgtaggacgacaccatgaacatgaagaagaggaagaggacggtaggaccagctgcagcagcagctacttttcctgagctgccggaggagattgtgatggagattctggctcgtctgcccgtcaaatccctgctacgcttcaagtccgtctgcagagggtggcgtgcaatcatctccgagccctccttcatcccaggggcggagctacagtgatgcctatgtattcccaggaatacccaactttttttatcaaaaaacaactatacttcaatatatacacataacacatatacacatattagttgtccccactcctccagagtccctcgctcggctcacgcctcaccctcacgcgcaaatgcgctgctcgcccgctccccaattccccatttgattccaaaccctaggtggcggcgttcggcggcggcgcggcggcgcggcgaccggcggaagcggcaaggcgtccggcgaccggcgacgggagcagggagctagaccagcggtggcgcggcgaggcattcggcagcggcgcgacgccgtggcgaggcattcggcggcggcgcggcgaggccgcgagacggcaaccgacgggcgaccggtgacggcgggagcgaggccacgaggccgaggggcaggggcgcggcggcggccggccaacagctgcagctcagcgggacagcgcccagcggcactgcggcaggcacaatttcaggtgacaaatcaagagttcaagattcaatcagtcctaaatccactcactatgtgtccatgtctgatgtttttttaccttagtctctgagtagtttctttttcctgtacttttgtagttctgtagattgaagaactctcaaaatgaagaagaaatcgattgatttgaagactttgtgggatagacattcaaagtctaggaaggtagggttaggttctggttctggatcaacaacacagccagttagaattgacagtgaagttgctgttccttctgttcagccgattacagatgcaaatgagagtccagttcagattcagattactgcagttgaggttgtgcctgaggtagcagccagtgatgcagcaagagatgtgacagaacctgaaacagaggtagctagcaatgatccagacatagcaacagcagtgacacagcaggagcaaccttccacttggtctcctatcagagattggtctcccatcagagatggtgatgatgaagagacagagtatgactcaagtgatgaagctatttatgacattgatttactttgtcatgatcctggaaggcgaattgcaatcaaaaattatgatgtcaatgaacggaattctgtgataaggggatatattgcattagggccatgccaaccacgcagccacagttttcctataaggaaaattggaggtaagcctcggcgcttccttcctagttggtatgacgaatttaaatggcttgaatatagtgtggaacaagatgctgcattctgttttatttgttacttattcaagcataaaattaataattctggtggagatgcatttgtaaataggggatttaggaattggcacatgagaaaaaggattgcaaaacatgttggtggtatgactagctttcacaatgtggcacaagataaatacaatcacttccttgcacctaaaacaaatattgttgagagctttgctgccaccaatgaacaagacaaggctagatatatggctcgtttaacctattcaatgaagtgcttgaagtttcttttaaggcaaggtttggctgcccgtggacattatgaaagtgaaaagtcacttaataaaggaaatttccttgagatgttgagtatgctagcagaaaactttgaagaagttggtaaggtggttttgaataatgctccaaagaattgtaagttgactgctcccgaaatacaaaaacagatagctaattgttgtgccaaagaaactactaagcttatcatggaagaccttggtgatgaatattttgcaatacttgccgatgaatctagtgatgtgtaccaaaaagaacagttggctctttgtttgagatatgttgataaaaaaggaagggtagttgagaggttccttggtgttgttcatgttgaaaatactacttccttgacacttaaagctgcaattgaatcattgcttatggagcattccttgagcttgtctaaggtccgtgggcaaggctatgacggtgctagtaacatgaagggtcatgctaatggactaaagaaattgattatggatgagtgcccttctgcctattatgtccattgctttgcacatcaactccaattaacacttgtggctgttgctaaggaaaacccagattgtgtgtggttctttgagcaacttagatttttgttaaatcttcttgggaattcttgtaagaagacagaaatgcttagagttgcacaagctcaaagaattgtagaagaactagatttgggtgacattgaaactggaaagggtttgaatcaagaaatgggtttgggcaggccagcagatactcgttggggatctcactataaaactgttatgcatgtcctatctttgtatccttcaattcaaaaagttcttataatggttggcaaggatcgctcttttggtgcagaatgtgcaaatgcacaaacagtgttgacaatattccaatcatttgagtttgtttttatggcacacttgatgcaaacagtacttgggttcacatctgacttgaatcatgctttgcagaagagggatcaagacattgttaatgcagtaggactgattttattgacaaagtttcaattgcagcaattacgcgaggatcctggatgggatgattttcttcaagaagtgcaatctttttgtgtgaagcataagatcaagatccctgatatggactctttctatcggccggttggaagagataggaggttctttattaaaatcaagaatatgcatcgcttccatgttgacatgtttctaagtgtcattgatagacaactacaagagcttaatgaacggtttgatgaggtaaacacagatttgctcctttgtatggctgcatttagtcctatagataactttgctagttgggacaaagataagttgattaagcttgcacggttttatcctaatgatttctcaagcacagagatgaaccatcttccatcagcattgaaactcttcctcactgagatgcgtatagatgaaaggtttagaaaagtaaaaaatcttgctaatctttccattatgattgttgaaacaaaattgcacaatagacatgagattgtttacaagcttctcaaattggttctggtacttccagtagcaacagctagtgttgaaagaatattttctgccatgaattatgtgaagaataaattgagaaacaggatgggggatcaatacttgaatgattgcttggtcacatttattgagcgtgagatgtttttgaaagtcaaggagtgtgatattataaaccgctttcaagccatgaaggaacgcagaattaaagctactcttccaagccacgaaggaaccgaaatagaaaattagaggtaccatgtaataatttgtaattttatttcttagattatcggtattgacatactgtgaaccattgattttttttactatgaatttttttgttagacgggaatacccaactttcaaatcctggctccgccactgcttcatccgaacccaactccagtgctcggcttccagacggcagcaggagccatccatcctcatcagcccccacttccgccatgaccggccgaccaaattctccacccacatcagcttctaccagtgggagcaaggtgcctcctcccttgcgaggatc

SEQ ID NO: 6 - DNA, 885 bp, Oryza sativa; identical to SEQ ID NO: 3
  source 1..885: mol_type = genomic DNA

SEQ ID NO: 7 - DNA, 885 bp, Oryza sativa; identical to SEQ ID NO: 3
  source 1..885: mol_type = genomic DNA

SEQ ID NO: 8 - protein, 294 aa, Oryza sativa; identical to SEQ ID NO: 4

SEQ ID NO: 9 - DNA, 22 bp, synthetic construct
  source 1..22: mol_type = other DNA
cttaccaccatatctgaatcac

SEQ ID NO: 10 - DNA, 18 bp, synthetic construct
  source 1..18: mol_type = other DNA
gctgatgaggatggatgg